(19) United States
(12) Patent Application Publication
(10) Pub. No.: US 2006/0287630 A1
(43) Pub. Date: Dec. 21, 2006

(54) Injection device with a torsion spring drive

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/444,897

(22) Filed: Jun. 1, 2006

(30) Foreign Application Priority Data
Jun. 2, 2005 (DE) 10 2005 025 424.1

(51) Int. Cl. A61M 5/20

(52) U.S. Cl. 604/130

(57) ABSTRACT

An injection device including a torsion spring with a mid-axis and an output element with a longitudinal axis which can be displaced along its longitudinal axis by the torsion spring, the mid-axis of the torsion spring being generally parallel with the longitudinal axis of the output element.

### CROSS-REFERENCE TO RELATED APPLICATION

This application claims the benefit of German Application No. 10 2005 025 424.1, filed on Jun. 2, 2005, the content of which is incorporated herein in its entirety.

### BACKGROUND

The present invention relates to device for delivering, administering, dispensing or injecting substances, and to methods of making and using such devices. More particularly, it relates to an injection device, more particularly an automatic injector or an injection pen, and a method whereby an output element for dispensing a product is driven by a torsion spring.

Injection devices are known from the prior art, by means of which what is usually a liquid product can be administered to a patient. Such injection devices typically have a plunger rod, which is driven forward by an appropriate drive and thus drives a plunger with it, which is mounted so that it can be moved in a product container. A movement in a dispensing direction toward a needle or cannula forces the product out of the container.

In many injection devices, a spring is used to provide a drive force. Depending on their spring path, springs have different spring forces. For example, a spring which is pushed back across a large spring path will have a larger spring force than the same spring pushed back across a shorter spring path. Since the spring force of springs is dependent on their path, problems can arise if a plunger rod of an injection device is required to exert as constant as possible a force on the product to be dispensed. Generally, as a result of the spring force, the plunger rod exerts a different force on the plunger at the start of dispensing the product than it does at the end of dispensing the product.

U.S. Pat. No. 5,478,316 discloses an automatic injection device which has a strip-shaped, spiral-shaped coiled spring, which acts on a plunger rod by means of its peripheral end. In order to effect a biasing action, the coiled spring, fixedly connected to a housing at one end, is rolled so that the coiled part of the spring extends along the plunger rod. Since the spring rolls back into the relaxed position, the plunger rod is driven forwards by the coiling peripheral end of the spring to dispense a product.

### SUMMARY

One object of the present invention is to provide a compact injection device. Another object is to provide a method by which a product can be efficiently dispensed.

The invention relates to injection devices, for example an injection pen or an automatic injector. An injection pen is an injection device of an elongate design which contains a product container or in which a product container can be inserted. The container contains a usually liquid product, and the injection pen comprises a mechanism enabling the product to be metered in a dose and/or dispensed. An automatic injector comprises an embodiment of an injection pen. Specifically, with an automatic injector, a product contained in the automatic injector is administered automatically when a mechanism is triggered. In an automatic injector, a needle may be automatically injected into the skin of a patient before dispensing the product. The process of injecting the needle followed by dispensing of the product may take place under the control of a suitable sequence control system.

In one embodiment, an injection device in accordance with the present invention incorporates a torsion spring with a mid-axis. The torsion spring may be a torsion bar, a spiral spring, a coil spring, a suitable combination of types of springs, a combination of a coil and spiral spring, or other suitable bias generating structure. In one preferred embodiment, a spiral spring may be made from a simple wire or a strip-shaped spring material, e.g. spring steel. The term “mid-axis” is intended to mean the axis about which the spring is generally coiled. In the case of a torsion bar, this may correspond to its longitudinal axis. An advantage of a spiral spring is, for example, that the spring force varies only slightly across a large spring path.

An injection device in accordance with the present invention has an output element which can be moved along its longitudinal axis and can be driven by the torsion spring. In some embodiments, the output element and torsion spring may be coupled with one another. In some embodiments, the longitudinal axis of the output element is more or less parallel with the longitudinal axis of the injection device, or corresponds to the longitudinal axis of the injection device. The output element may be a plunger, longitudinally displaceable in a product container, at least moveable in forward or dispensing direction to dispense a product. For example, the output element may be driven by the torsion spring in such a way that the output element is moved in the longitudinal direction relative to the torsion spring. In some embodiments, the output element may be mounted or carried by a housing, so that it is not able to rotate. One of the output element and housing may have an engaging element which is able to engage in at least one complementary element of the other, as a result of which the output element is able to move longitudinally and is prevented from turning. A drive element is coupled with the output element so that a rotating movement of the drive element is able to generate the longitudinal movement of the output element. The longitudinal axis of the drive element may be more or less parallel with the longitudinal axis of the output element or may correspond to the longitudinal axis of the output element. The drive element may be mounted on or carried by the housing so that it is able to rotate but is axially fixed. The drive element may be axially fixed relative to the torsion spring and/or the housing. In some embodiments, one of the drive element and output element may have an engaging element able to engage in the other and convert the rotating movement of the drive element into a longitudinal movement of the output element. The drive element and output element may be coupled by a threaded drive, for example. The drive element and output element may each have a thread, in which case the thread of one engages in the thread of the other.

In some embodiments, the mid-axis of the torsion spring is more or less parallel with the longitudinal axis of the output element. The mid-axis of the torsion spring may therefore also be more or less parallel with the longitudinal axis of the drive element or the injection device. In some preferred embodiments, the mid-axis of the torsion spring may correspond to the longitudinal axis of the injection device, the drive element or the output element or may be one of these longitudinal axes. In some embodiments, it may be preferable for the engagement cam or threaded drive to have a pitch which is not self-inhibiting for a force acting in the circumferential direction and/or in the longitudinal direction. For example, the output element may be pushed with an external force acting on the output element in the longitudinal direction so that the torsion spring is tensed. In some embodiments, after an injection has taken place, the output element may be pushed back into its initial position, causing the drive element to be displaced in rotation and thus tense the torsion spring. The housing and drive element are coupled by means of the torsion spring. In some embodiments, a first end of the torsion spring may be fixedly connected to the housing and a second end of the torsion spring may be fixedly connected to the drive element. The torsion spring may be biased forward as the drive element rotates. The torsion spring transmits the energy stored in it due to the pulling action to the drive element, in which case the drive element is displaced in rotation. The drive element may be supported in the longitudinal direction distally and the torsion spring proximally in the radial direction. Alternatively, the drive element may also be clamped in a floating arrangement and the torsion spring may be connected to the drive element externally to the floating clamp arrangement.

In some preferred embodiments, an injection device in accordance with the present invention comprises a pulling means or mechanism which is rotatable in at least one direction of rotation, as a result of which the torsion spring can be biased. For example, a rotation is possible in one direction of rotation and prevented in the other direction of rotation. The pulling means may be coupled with the torsion spring in such a way that, for example, the pulling means is able to bias the spring both when rotating in the one direction of rotation and when rotating in the other direction of rotation. In some preferred embodiments, when the pulling means is moved in a first direction of rotation, the torsion spring is tensed and when the pulling means is moved in a second direction of rotation, the torsion spring is not relaxed. For example, the pulling means may be coupled with the torsion spring by a so-called ratchet mechanism so that it pulls on the spring when rotated in a first direction of rotation and does not pull on the spring when rotated in the second direction of rotation. The pulling means and a catch means may cooperate in a catch engagement so that the pulling means drives the catch means with it when rotated in a first direction of rotation, and the pulling means is turned relative to the catch means when rotated in the other, direction of rotation. In some preferred embodiments, a rotating movement of the pulling means in at least one direction of rotation may be transmitted to the drive element. The pulling means and/or the catch means may be of a sleeve-shaped design and/or comprise, for example, a body with a generally central opening. In some preferred embodiments, one of the catch means and pulling means engages in the other so that a movement of the pulling means relative to the catch means is possible in one direction of rotation and is not possible in the other direction of rotation. For example, the catch means may be driven with the pulling means in one direction of rotation and not driven with it in the other direction of rotation.

In some embodiments, one of the pulling body and catch may have at least one cam, which is able to engage in the other of the pulling body and catch, in a recess provided thereon. The at least one cam may be disposed on the pulling body pointing toward the catch or on the catch pointing toward the pulling body. The at least one cam may be resiliently disposed so that it is able to spring into and/or out of recesses. The at least one cam may move more or less radially with respect to the mid-axis of the pulling body or catch. For example, the at least one cam may be disposed respectively on an arm which extends more or less in the peripheral direction and may be disposed on one of the catch or pulling body. The at least one cam may be designed so that when the pulling body rotates in a first direction of rotation, it drives the recess with it, whereas in another, second direction of rotation, it is pushed out of the recesses, away from the recess. The at least one cam may be of a sawtooth shape, in which case it has a surface inclined more or less perpendicular to the peripheral direction which is able to lock the relative rotation between the pulling body and catch, and has a surface inclined at an acute angle which can be pushed by the recess away from the recess. The at least one recess may be adapted to the shape of the cam. The number of the at least one recess may correspond to a multiple, e.g. twice, the number of the at least one co-operating cam.

In some preferred embodiments, the catch means and the housing are in a catch engagement so that the catch means can not rotate in a first direction of rotation but can rotate in a second direction of rotation. For example, one of the catch means and housing has at least one locking cam which is able to engage in the other of the catch means and housing in a recess. The at least one locking cam may be disposed on the catch means, pointing toward the housing or on the housing pointing toward the catch means. In some embodiments, the at least one locking cam may be of a resilient design so that it can spring into and/or out of the recess. The at least one locking cam may be resilient more or less radially with respect to the mid-axis of the catch means or housing. For example, the at least one locking cam may be disposed respectively on an arm extending in the circumferential direction and on one of the catch means or housing. The at least one locking cam may be designed so that it can prevent a rotation of the catch means relative to the housing in one, for example the second, direction of rotation and when the catch means is rotated in another, for example the first, direction of rotation, it is pushed out of the recess, away from the recess. The at least one locking cam may be of a sawtooth-shaped design, in which case it has a surface inclined more or less perpendicular to the circumferential direction which is able to block a relative rotation between the catch means and housing, and a surface inclined at an acute angle which can be pushed by the recess out of the recess. The at least one cam may be disposed so that it acts between the pulling means and catch means and the at least one locking cam acts between the catch means and housing in opposite directions of rotation.

In some embodiments, the catch means may engage around the drive element, and is disposed in a releasable engagement with the drive element. When the catch means is engaged with the drive element, the catch means may be prevented from rotating relative to the drive element and/or the catch means can be displaced longitudinally relative to the drive element. For example, the drive element and catch means may be connected to one another by means of a multi-edge connection, a multi-tooth connection or other known positive connection, which prevents any mutual relative rotation but permits a relative displacement in the longitudinal direction. For example, the drive element may have a first portion which has an anti-rotation lock for the catch means and a second portion which does not have an anti-rotation lock for the catch means. The second portion may have a circular cross-section. The second portion may adjoin the first portion, for example proximally, in the longitudinal direction. The catch means may be pushed so that it moves along the longitudinal axis of the drive element from the first portion into the second portion so that the drive element is released by the catch means to rotate. The catch means may be pushed against the force of a spring, which pushes the catch means into the first portion. The at least one recess for the at least one cam or locking cam of the pulling element, catch means or housing may be grooves, for example, which extend in the direction of the longitudinal axis of the drive element so that the at least one cam or locking cam is able to slide axially along the recess.

In some embodiments, it may be preferable for an operating element to couple with the catch means so that the catch means can be moved out of engagement with the drive element when the operating element is moved. For example, to release the engagement of the catch means with the drive element, the operating element may be moved in a different direction from the catch means during release, e.g., the catch may be moved along and the operating element transversely to the longitudinal axis of the drive element. The operating element may project out from the housing or may be operated by the user through the housing. The operating element may be a button on other suitable structure. The operating element may be coupled with the catch means by means of a sliding element which can be moved more or less in the longitudinal direction of the device. The sliding element may extend at least partially around the torsion spring. When the operating element is moved transversely to the longitudinal axis of the injection device, the sliding element may move ore or less parallel with the longitudinal axis of the injection device. The operating element and/or the sliding element may each have a gear element, in which case the gear elements co-operate so that the sliding element is moved more or less parallel with the longitudinal axis of the injection device. The gear elements may be surfaces, such as flat surfaces, for example, which are able to slide on one another. The surfaces may be disposed transversely to the directions of movement of the sliding element and the operating element. When the operating element is operated, its gear element may be moved into engagement with the gear element of the sliding element, in which case a further movement of the operating element will push the sliding element toward the catch means in order to move the catch means out of the positive engagement with the drive element.

In some embodiments, it may be preferable if the catch means can not be released unless the sliding element is in a release position or is moved from an initial position into a release position. For example, the gear elements of the operating element and the sliding element may be moved into engagement in the release position and are not in engagement in the initial position. The sliding element to be retained in the initial position by means of a spring and moved from the initial position into the release position against the force of the spring.

In some embodiments, the sliding element may be coupled with a guard element, such as a needle guard sleeve, which is able to move the sliding element from the initial position into the release position during a movement to release a needle. The guard element may be mounted by means of a housing of the device or an ampoule holder in which the product container is inserted or can be inserted. The guard element may be moved by means of a spring, for example, into a position so that the sliding element is able to assume its initial position. The guard element may be pushed in the direction of the sliding element, so that the guard element moves the sliding element from the initial position into its release position. This ensures that product can only be dispensed by means of the device when the guard element is placed on a tissue of a patient, and pressed against it.

In one embodiment of the present invention, a product container which is inserted in or can be inserted in the device can be moved in the dispensing direction by the output element. For example, the product container, such as an ampoule, may be mounted by means of a housing and an ampoule holder so that it can move longitudinally. The distal end of the container may carry a needle which can be injected into a body tissue and through which the product can be injected from the container into the tissue of the patient.

In some embodiments, to prepare for dispensing a product, the torsion spring may be biased by rotating the pulling means. When the pulling means is rotated in a first and/or a second direction of rotation, the rotating movement is firstly transmitted to the catch means and from the catch means to the drive element. The torsion spring, which in some embodiments is preferably fixedly joined to the drive element, is pulled or biased forwards by the rotating movement of the drive element. Once the torsion spring has been biased forwards, it is not able to relax, due to the catch engagement of the catch means with the drive element and the housing. The needle can now be injected into the tissue of the patient and/or the guard element placed on the tissue so that the needle is injected or not injected if the product container is longitudinally displaceable. In some embodiments, it may be preferable if, when the sliding element is in the release position, the operating element is operated by the user, causing the sliding element to be pushed in the direction of the catch means. Due to the displacement of the sliding element, the catch means is pushed axially far enough out of the positive engagement formed by the catch means with the first portion of the drive element so that the catch means moves out of engagement with the first portion of the drive element and into the second portion of the drive element. When the catch means is no longer in engagement with the drive element, it has reached its release position. In some embodiments, the catch means has released the drive element when it reaches the release position so that the torsion spring can relax and thus drive the drive element. The torsion spring is able to drive the drive element in a direction of rotation that is opposite the direction of rotation of the drive element in order to pull on the spring. As the torsion springs transmits the driving movement to the drive element, the output element is moved in the dispensing direction. The output element may act on the plunger of the product container, to force a liquid product contained in the product container and injects it through the needle into the tissue of the patient. If the product container is longitudinally displaceable, the product container can be pushed in the injecting direction of the needle by the drive element acting on the plunger due to the viscosity of the product so that the needle is firstly injected into the tissue of the patient before the plunger is able to force the product out of the product container.

In some embodiments, a damping element may be provided for driving the torsion spring, which damps the driving action of the output element. As a result, the speed at which the output element is fed or moved forward and/or the force by which it is fed forward can be kept more or less constant for products of differing viscosities. For example, in the case of a product with a low viscosity, overshooting of the spring or too rapid a dispensing of the product is prevented. Means may be provided between the torsion spring and output element which are able to reduce and/or control the driving force and/or the driving speed of the torsion spring toward the output element.

The present invention encompasses a method of dispensing a product contained in an injection device. Accordingly, an output element is moved in a direction such that the product is dispensed. This direction may point in the longitudinal direction of the device or the output element, for example. The longitudinal axis of the output element is enclosed by a torsion spring, the spring energy of which is transmitted to the output element. The torsion spring may be directly or indirectly coupled with the output element, i.e. via another element.

In some preferred embodiments, the catch means releases the spring with a movement along the longitudinal axis of the output element in readiness for dispensing a product. In some embodiments, the container containing the product is also driven along by a movement of the output element. In some preferred embodiments, the product contained in the container may be dispensed by the device in accordance with the method in one operation once dispensing has been triggered.

### BRIEF DESCRIPTION OF THE DRAWINGS

FIG. 1 is a sectional view illustrating an embodiment of an injection device in accordance with the present invention,

FIG. 2 is a sectional view along line A-A indicated in FIG. 1,

FIG. 3 is a sectional view along line F-F indicated in FIG. 1,

FIG. 4 is a sectional view along line C-C indicated in FIG. 2,

FIG. 5 is a sectional view along line E-E indicated in FIG. 2,

FIG. 6 is a sectional view along line D-D indicated in FIG. 2,

FIG. 7 is a sectional view along line G-G indicated in FIG. 2, and

FIG. 8 is an exploded assembly view of the embodiment illustrated in FIGS. 1 to 7.

### DETAILED DESCRIPTION

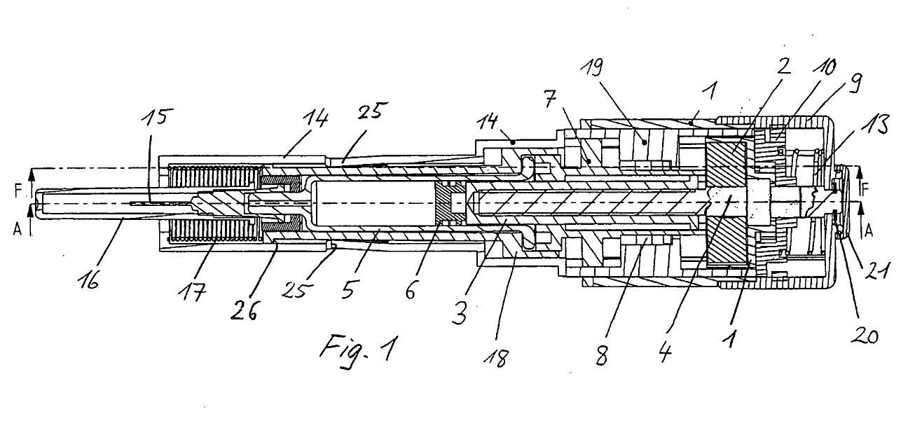

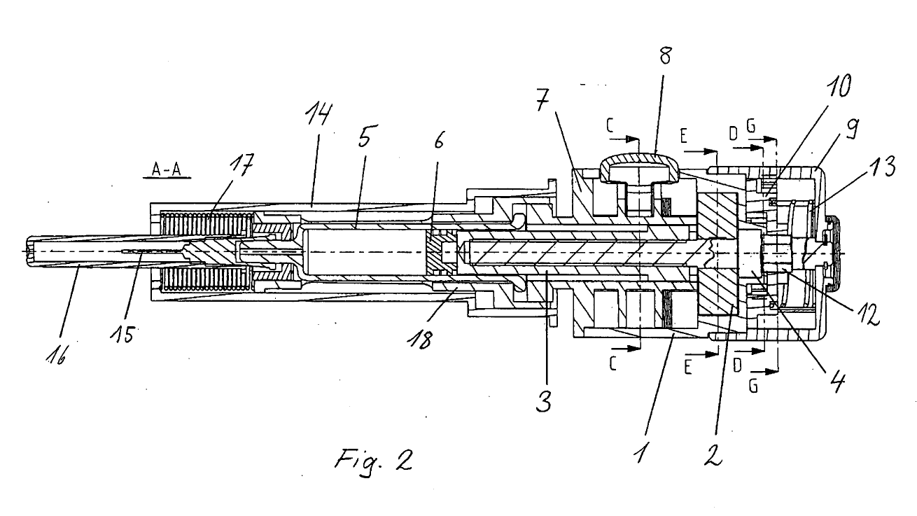

FIGS. 1 to 7 illustrate one embodiment of an injection device in accordance with the present invention in the form of an injection pen in an initial position. The injection device incorporates a torsion spring 2, which is fixedly connected to a housing 1 by its first end and to a drive element 4 by its second end. During a relative rotation between the drive element 4 and housing 1, the spring is relaxed or tensed. The spring 2 may be made from a strip-shaped material, e.g., spring steel, and is wound to form a spiral. The housing 1 mounts or carries the drive element 4 so that it is rotatable but axially fixed. The housing 1 also extends round the torsion spring 2, at least partially around its circumference, and forms a catch engagement in conjunction with a catch 10.

The drive element 4 is coupled with an output element 3 by means of a threaded drive. The output element 3 is axially displaceable by means of a mechanism holder 7 and is mounted so that it can not rotate. The mechanism holder 7 is connected to the housing 1 so that it can not rotate and is axially fixed. When the drive element 4 is rotated, the output element 3 is displaced in a movement along its longitudinal axis. The distal end of the output element 3 hits a plunger 6, which is mounted so as to be displaceable in a container 5 for a liquid product. When the output element 3 is moved in the direction of the plunger 6, the plunger 6 is pushed in the dispensing direction and the product contained in the container 5 is dispensed via a needle 15 disposed on the distal end of the container 5.

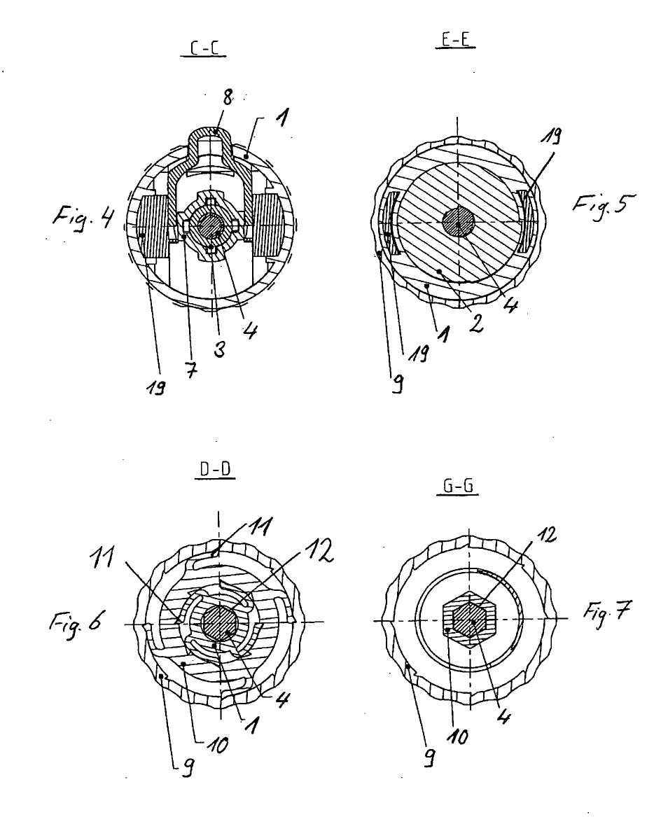

In some embodiments, the container 5 is accommodated by an ampoule holder 18. The ampoule holder 18 is attached to the mechanism holder 7, e.g. screwed thereto. As may be seen in particular form FIG. 4, the mechanism holder 7 has four grooves extending in the longitudinal direction, in each of which a cam formed by the output element 3 engages, forming an anti-rotation lock and a longitudinal guide for the output element 3.

The catch 10 couples the housing 1, drive element 4 and pulling body 9. In some embodiments, the pulling body 9 is a generally tubular member or sleeve closed at an end face mounted so as to be rotatable 1 relative to the housing 1. At its terminal face disposed at the proximal end, the pulling body 9 has a bore concentric with the longitudinal axis of the drive element 4. The drive element 4 extends through this bore and has an axial locking ring 20 so that the pulling body 9 is axially fixed relative to the drive element 4 at this point and mounted so that it can rotate. A cap 21 may be placed on the pulling body 9, which covers the axial locking ring 20 and the proximal terminal face of the drive element 4.

As may be seen in particular in FIG. 6, the catch 10 is designed so that when the pulling body 9 is rotated clockwise, the pulling body 9 can be rotated relative to the catch mans 10. When the pulling body 9 is rotated anti-clockwise, the pulling body 9 and the catch 10 are turned together relative to the housing 1. The pulling body 9 and the catch 10 each have recesses on their inner circumference, which are adapted to match cams 11a or locking cams 11b of a sawtooth design and in which the cams 11a, 11b can engage. To engage in the recesses of the pulling body 9, the cams 11a are provided in the form of catch 10. The cams 11b for the recesses of the catch 10 are formed by the housing 1. The cams 11a, 11b each sit at the end of an arm disposed on the peripheral surface of the catch 10 or the housing 1. The cams 11a, 11 b may be resilient more or less about the fixing point of the arm with the catch 10 or housing 1. During the clockwise rotation, the cam 11a of the catch 10 is pushed by the surface of the recess subtending an acute angle with the circumferential direction of the pulling body 9 out of engagement with the recess. The cam 11b of the housing 1 prevents the catch 10 from rotating in the clockwise direction because the cam 11b is blocked against a surface disposed in the anti-clockwise direction more or less at a right angle with respect to the circumferential direction of the catch 10. During a rotation in the anti-clockwise direction, the cams 11a of the catch 10 block a rotation of the pulling body 9 relative to the catch 10, so that the catch 10 is driven along by the pulling body 9. The cams 11b of the housing 1 are forced by the recesses out of the recesses due to the rotating movement of the catch 10 in the anti-clockwise direction so that the catch 10 is able to rotate relative to the housing 1. The pulling body 9 and the catch 10 have a multiple, e.g. two times, more recesses than respective cams 11a, 11b on the external periphery of the catch 10 or housing 1.

As illustrated in FIG. 7, the catch 10 is positively connected to the drive element 4 so that it is locked with it rotation. To form the rotationally locked connection, the catch 10 has an internal hexagon head and the drive element 4 has a matching external hexagon head. When the device is in the position illustrated in FIGS. 1 to 7, a rotation of the pulling body 9 causes the torsion spring 2 to be pulled or at least not relaxed. When the torsion spring 2 is pulled on or biased, the torsion spring can not relax and can therefore not drive the drive element 4 because the drive element 4 is prevented from rotating by means of the hexagon head connection to the catch 10 and the engagement of the housing 1 in the catch 10.

The recesses of the pulling body 9 and/or the pulling means 10 for the cams 11a, 11b extend more or less in the longitudinal direction of the drive element 4. The cams 11a, 11b may therefore slide along, in or on the recesses, in the longitudinal direction. The catch 10 is able to move along the longitudinal axis of the drive element 4, in which case the rotationally fixed engagement of the catch 10 with the drive element 4 is released once pushed in the proximal direction into the second portion, which does not have a hexagon head. Disposed between the pulling body 9 and the catch 10 is a spring 13, which holds the catch 10 at least in the initial position and, in some preferred embodiments, also in the release position, in the position illustrated in FIGS. 1 to 3.

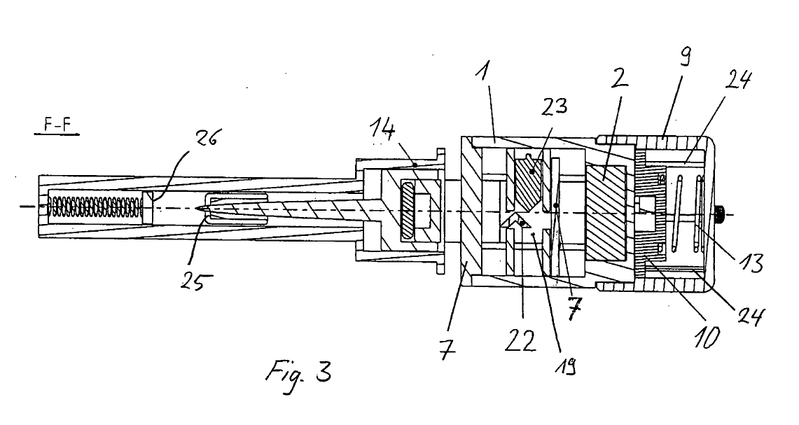

The catch 10 may be pushed out of the rotationally locked engagement with the drive element 4 by means of a sliding element 19 accommodated in the housing so that it can be displaced longitudinally. As illustrated in FIG. 5, the sliding element 19 has a fork-shaped portion, which extends through the housing 1 past the peripheral face of the torsion spring 2. The proximal end of the sliding element 19 may abut with the catch 10 and push it against the force of the spring force 13 in the proximal direction. As may be seen in particular from FIG. 3, the sliding element 19 has a gear element 22. The gear element 22 has two surfaces inclined toward the longitudinal axis of the device. One of the two surfaces points in the distal direction and the other in the proximal direction. The surfaces may be inclined by approximately 45° toward the longitudinal axis of the device. The device has an operating element 8, which is displaceable transversely, in some embodiments perpendicular to, the longitudinal axis of the device. As illustrated in particular in FIG. 4, the operating element 8 is fork-shaped and is able to engage laterally around the drive element 4 and/or the output element 3. On its two fork ends, the operating element 8 has a respective gear element 23, which is able to co-operate with a gear element 22 of the sliding element 19 associated with it. The gear element 23 of the operating element 8 has two gear surfaces extending transversely, e.g. at an angle of 45°, with respect to the longitudinal axis of the device. One gear surface of the gear element 23 points in the distal direction and the other in the proximal direction. The two mutually inclined gear surfaces of the gear element 23 and the gear element 22 each form tip for each gear element 22, 23 at the point at which the gear surfaces converge. In the initial position, as illustrated in FIGS. 1 to 7, the tip of the gear element 22 of the sliding element 19 is disposed distally in the longitudinal direction of the device with respect to the tip of the gear element 23 of the operating element 8. When the operating element 8 is operated, namely when moved transversely to the longitudinal axis of the device, the gear surface of the gear element 23 pointing in the distal direction and the gear surface of the gear element 22 pointing in the proximal direction move into engagement. The sliding element 19 is moved in the distal direction, or at least the sliding element 19 is not moved in the proximal direction. In spite of the fact that the operating element 8 has been operated, the catch 10 is not moved out of its position illustrated in FIG. 3. In order to be able to move the catch 10 out of the position illustrated in FIGS. 1 to 7, the sliding element 19 is pushed so far in the proximal direction that the tip of the gear element 22 of the sliding element 19 reaches a position which is proximal with respect to the tip of the gear element 23 of the operating element 8. The gear element 22 of the sliding element 19 must be pushed at least far enough in the proximal direction to enable the gear surface of the gear element 23 pointing in the proximal direction to engage with the gear surface of the gear element 22 pointing in the distal direction. In this so-called release position, a movement of the operating element 8 transversely to and with respect to the longitudinal axis causes a movement of the sliding element 19 in the proximal direction because the gear surfaces of the gear elements 22, 23 convert the transverse movement into the longitudinal movement. This being the case, the catch 10 is pushed in the proximal direction. When the catch 10 has been pushed so far that it moves out of the positive anti-locking position with the drive element 4, the drive element 4 is released so that it can rotate. The torsion spring 2 drives the drive element 4 onward, and the rotating movement of the drive element 4 is converted via the threaded drive into a longitudinal movement of the output element 3. The output element 3 acts by means of its distal end on the plunger 6, which forces the product contained in the container 5 and dispenses it via the needle 15.

As illustrated in FIG. 1, for example, the sliding element 19 may be moved from the initial position into the release position by means of a guard or cover 14. The guard 14 may be a needle guard sleeve, for example. The needle guard sleeve 14 is mounted by means of the ampoule holder 18 so as to be longitudinally displaceable. At its distal end, the guard 14 has an opening, through which the needle 15 can extend. By way of example, the guard 14 is illustrated in a partially operated position in FIG. 1, in which the guard 14 has been pushed far enough back in the proximal direction that the sliding element 19 still remains in its initial position. When the guard 14 is pushed farther in the distal direction, the sliding element 19 is moved out of its initial position into the release position. To this end, the proximal terminal end of the guard means 14 acts on the distal terminal end of the sliding element 19. In a non-operated state, the guard means 14 is pushed so far in the distal direction by the spring 17 that the terminal end of the catch lug 25 moves into contact with the stop 26. This being the case, the needle 15 is covered to the degree that a user of the device can not inadvertently pierce himself. When the guard means 14 is placed on the skin of a patient, for example, and pressure is applied, the guard means 14 is pushed in the proximal direction and the needle 15 is injected into the skin. The device therefore ensures that a product can not be dispensed until the needle 15 has penetrated the body tissue by a certain degree.

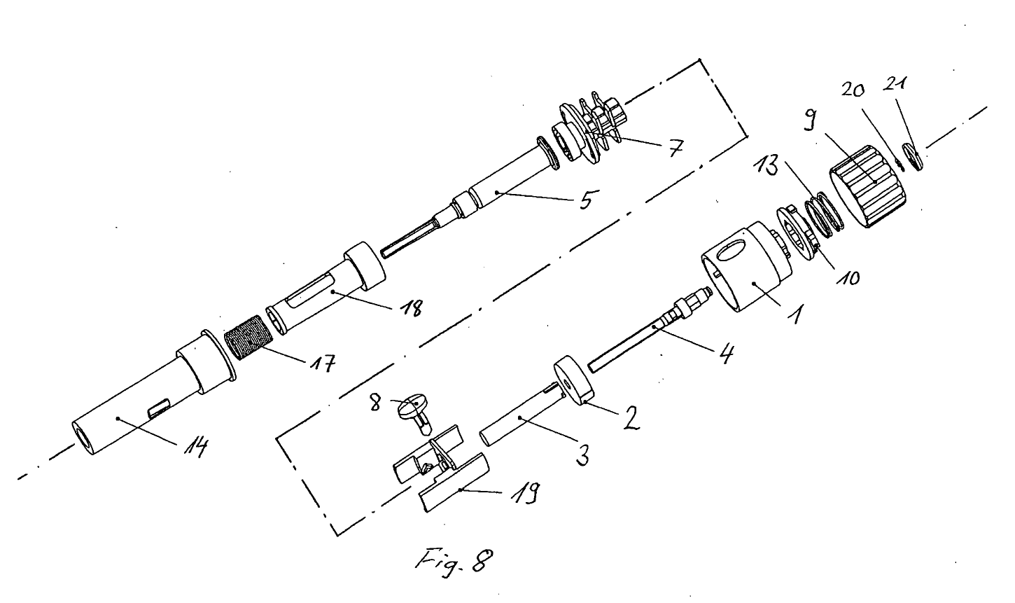

FIG. 8 provides an illustration showing the features of the individual components of the injection device described with reference to FIGS. 1 to 7.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising:
a) a torsion spring with a mid-axis, and
b) an output element having a longitudinal axis, said output element moveable along the longitudinal axis by the torsion spring, wherein the mid-axis of the torsion spring is generally parallel with the longitudinal axis of the output element.

2. The injection device as claimed in claim 1, further comprising a body rotatable in at least one direction of rotation for biasing the torsion spring.

3. The injection device as claimed in claim 2, further comprising a product container moveable in a dispensing direction by the output element.

4. The injection device as claimed in claim 3, further comprising a drive element coupled with the output element such that a rotating movement of the drive element generates a longitudinal movement of the output element.

5. The injection device as claimed in claim 4, wherein the drive element and the output element are coupled by one of a threaded drive or an engaging cam.

6. The injection device as claimed claim 5, wherein the engaging cam or the threaded drive has a pitch which is not self-locking whereby the output element is moveable in such a direction that the torsion spring is tensed by a force acting in the longitudinal direction.

7. The injection device as claimed in claim 6, wherein when said body is moved in a first direction of rotation, the torsion spring is tensed, and when the body is moved in a second direction of rotation, the torsion spring is not relaxed.

8. The injection device as claimed in claim 1, further comprising and a sleeve and a catch, said sleeve and catch in a catch engagement so that the sleeve drives the catch with it when rotated in a first direction of rotation and is turned relative to the catch when rotated in a second direction of rotation.

9. The injection device as claimed in one claim 8, wherein the rotating movement of the sleeve is transmitted to a drive element in at least one of the directions of rotation.

10. The injection device as claimed in claim 9, wherein the catch is in a releasable engagement with the drive element.

11. The injection device as claimed in claim 10, where the catch is axially moveable relative to the drive element.

12. The injection device as claimed in claim 11, further comprising an operating element coupled with the catch so that the catch can be moved out of engagement with the drive element when the operating element is moved.

13. The injection device as claimed in claim 12, wherein the operating element is coupled with the catch via a longitudinally displaceable sliding element.

14. The injection device as claimed in claim 13, wherein the operating element is moveable transversely to the longitudinal axis of the injection device and the sliding element is moved by the operating element generally parallel to the longitudinal axis of the injection device.

15. The injection device as claimed in claim 14, wherein the catch can not be released unless the sliding element has been moved out of an initial position into a release position.

16. The injection device as claimed in claim 15, further comprising an axially displaceable needle guard sleeve coupled with the sliding element so that when the needle guard sleeve is moved in a direction opposite an injection direction of the injection device, the sliding element is moveable into the release position.

17. The injection device as claimed in claim 16, wherein the release position the catch has released the drive element so that the torsion spring can relax and the drive element can be driven.

18. The injection device as claimed in claim 17, further comprising a damping element for damping the driving action of the output element.

19. The injection device as claimed claim 17, further comprising means between the torsion spring and output element, for regulating at least one of the driving force of the torsion spring and the speed at which the spring is driven toward the output element.

20. A method of dispensing a product contained in an injection device, said method comprising the steps of moving an output element in a direction to dispense the product, and wherein the longitudinal axis of the output element is enclosed by a torsion spring, and the spring energy of which is transmitted to the output element.

21. The method as claimed in claim 20, wherein the injection device comprises a catch, the catch releasing the torsion spring to move along the longitudinal axis of the output element carrying the enable product to be dispensed.

22. The method as claimed in claim 21, further comprising the step of moving a container by the movement of the output element.
